(19) United States
(12) Patent Application Publication
(10) Pub. No.: US 2024/0148640 A1
(43) Pub. Date: May 9, 2024

(54) TOPICAL OXYBUTYNIN FOR HOT FLASHES

(71) Applicant: CELISTA PHARMACEUTICALS LLC, Edina, MN (US)

(72) Inventor: Yogesh DANDIKER, Edina, MN (US)

(21) Appl. No.: 18/502,863

(22) Filed: Nov. 6, 2023

Related U.S. Application Data
(60) Provisional application No. 63/422,804, filed on Nov. 4, 2022.

(51) Int. Cl. A61K 9/00; A61K 31/222; A61K 47/10; A61K 47/12; A61K 47/14; A61K 47/32

(52) U.S. Cl. CPC A61K 9/0014; A61K 31/222; A61K 47/10; A61K 47/12; A61K 47/14; A61K 47/32

(57) ABSTRACT

The present disclosure provides a sprayable composition comprising about 1% w/v to about 20% w/v of oxybutynin, a penetration enhancer, at least 65% w/v of an aliphatic solvent, and a film forming excipient, wherein the film forming excipient has a solubility in water, at a pH between 1 and 10 and wherein the composition is a sprayable liquid solution that forms a protective and washable film when sprayed on skin The disclosure also provides a method of treating vasomotor symptoms, hot flashes, hyperhidrosis, osmiodrosis, incontinence, bladder spasms, pain following bladder surgery, or combinations thereof in a subject in need thereof, the method comprising topically administering to the subject the topical compositions as described herein.

### FIELD OF THE INVENTION

The present disclosure provides a composition comprising about 1% w/v to about 20% w/v of oxybutynin, a penetration enhancer, at least 65% w/v of an aliphatic solvent, and a film forming excipient. The disclosure further provides a method of treating hot flashes, hyperhidrosis, osmiodrosis, incontinence, bladder spasms, pain following bladder surgery, moderate to severe vasomotor symptoms caused in menopausal women, and particularly in breast cancer patients in need thereof, the method comprising applying the composition as a spray to the skin.

### BACKGROUND

Oxybutynin is a well-known potent muscarinic receptor antagonist used to treat symptoms of an overactive bladder such as incontinence or an urgent and frequent need to urinate. Recent studies in menopausal therapy and treatment of hot flashes show that oxybutynin reduced the frequency and intensity of hot flashes in women who were suffering frequent hot flashes, including breast cancer survivors who were receiving tamoxifen or aromatase inhibitors (Kligman et al., 2010). However, when administered orally, oxybutynin can cause adverse effects including dry mouth, constipation, diarrhea, blurred vision, dizziness, impaired urination, somnolence, blurry vision, dizziness, nervousness, nausea and impaired cognition largely due to oxybutynin's primary metabolite after first-pass hepatic metabolism, N-desethyloxybutynin (DEO) (McCrery, 2006). Additionally, the bioavailability of oxybutynin when administered orally is only around 10%, which reduces the effectivity of the treatment (Kennelly, 2010).

In order to bypass the hepatic first pass metabolism of oxybutynin and prevent the side effects of DEO that occurs when oxybutynin is administered orally, topical administration for the delivery of oxybutynin has been attempted. For example, U.S. Pat. No. 8,980,290 reports a formulation comprising oxybutynin that is substantially free of long chain fatty alcohols, long chain fatty acids, and long-chain fatty esters. U.S. Pat. No. 10,149,828 reports a transdermal patch incorporating an oxybutynin formulation. A metered dose transdermal spray formulation comprising 10% oxybutynin has been reported for the transdermal delivery of oxybutynin (Bakshi et al., 2008). Commercial formulations include the active ingredient oxybutynin: an oxybutynin transdermal patch (Oxytrol®) and an oxybutynin gel (Gelnique®). However, creams, gels and transdermal patches can be difficult to apply, are inconvenient, and can involve a messy application process. Patches are known to cause erythema of the skin over long-term use. Gels and creams are known for inadvertent transfer oxybutynin to others who may come in contact with these formulations. Further, none of the previous treatment methods have been demonstrated to be effective and safe in treating vasomotor symptoms in menopausal women.

### SUMMARY OF THE INVENTION

The present disclosure is directed to a composition comprising about 1% w/v to about 20% w/v of oxybutynin, a penetration enhancer, at least 65% w/v of an aliphatic solvent and a film forming excipient, wherein the film forming excipient has a solubility in water, at a pH between 1 and 10, and wherein the composition is a sprayable liquid solution that forms a protective and washable film when sprayed on the skin.

In some embodiments, the present disclosure is directed to a composition comprising about 1% w/v to about 8% w/v of oxybutynin, a penetration enhancer, at least 65% w/v of an aliphatic solvent and a film forming excipient, wherein the film forming excipient has a solubility in water, at a pH between 1 and 10, and wherein the composition is a sprayable liquid solution that forms a protective and washable film when sprayed on the skin.

In one embodiment, the present disclosure is directed to a composition comprising about 6% w/v of oxybutynin, a penetration enhancer, about 35% to about 45% w/v of an aliphatic solvent and a film forming excipient, wherein the film forming excipient has a solubility in water, at a pH between 1 and 10, and wherein the composition is a sprayable liquid solution that forms a protective, breathable and washable film when sprayed on skin. In some embodiments, the composition comprises about 7% w/v oxybutynin. In some embodiments, the composition comprises about 7% w/v oxybutynin.

In one embodiment, the present disclosure is directed to a composition comprising about 8% w/v of oxybutynin, a penetration enhancer, about 35% to about 45% w/v of an aliphatic solvent and a film forming excipient, wherein the film forming excipient has a solubility in water, at a pH between 1 and 10, and wherein the composition is a sprayable liquid solution that forms a protective, breathable and washable film when sprayed on skin. In some embodiments, the composition comprises about 7% w/v oxybutynin. In some embodiments, the composition comprises about 7% w/v oxybutynin.

In some embodiments, the penetration enhancer is 1-dodecylazacycloheptan-2-one, isopropyl myristate, octisalate, oleic acid, diethylene glycol monoethyl ether (Transcutol® P), or combinations thereof.

In some embodiments, the solvent is acetone, ethanol and/or isopropyl alcohol.

In some embodiments, the film forming excipient is a polyacrylate polymer or a cellulose polymer. In some embodiments, the film forming excipient is methacrylic acid and methyl methacrylate copolymer 1:1, methacrylic acid and methyl methacrylate copolymer 1:2, poly(butylmethacrylate-co-(2-dimethylaminoethyl)methacrylate-co-methyl methacrylate 1:2:1, hypromellose, hydroxypropyl cellulose, and ethyl cellulose. In some embodiments, the composition comprises about 4% w/v to about 7% w/v of the film forming excipient.

In one embodiment, the composition comprises octisalate and methacrylic acid and methyl methacrylate copolymer 1:1.

In some embodiments, the composition comprises a washability enhancer. In some embodiments, the washability enhancer is polyethylene glycol 400. In some embodiments, the composition comprises tromethamine.

In some embodiments, the composition has a water vapor transmission rate, as a fraction compared to a non-occluded control, not less than 0.50 over a 48 hour period, when dried on a porous substrate.

In some embodiments, the composition forms a barrier film in less than three minutes after application to the skin. In some embodiments, the composition forms a barrier film less than two minutes after application to the skin. In some embodiments, the composition forms a barrier film less than one minute after application to the skin. In some embodiments, the barrier film is capable of preventing transfer of oxybutynin to others, for example to partners or children.

In some embodiments, the disclosure provides a method of treating hot flashes in a human, the method comprising applying the composition as a spray to the skin of the human. In some embodiments, the composition is applied once within a 24 hour period. In some embodiments, the composition is application more than once within a 24 hour period. In some embodiments, the method comprises applying a composition comprising about 0.5% w/v to about 15% w/v oxybutynin. In some embodiments, the total dosing amount of oxybutynin can be adjusted by adjusting the total number of applications of the composition, e.g., 1 dose, 2 doses, 3 doses, 4 doses, 5 doses, 6 doses, etc. In some embodiments, the amount of oxybutynin administered to a subject can be adjusted by altering the frequency of administration, e.g., once daily, twice daily, thrice daily, etc. In some embodiments, the amount of oxybutynin administered to a subject can be adjusted by reducing the frequency of administration, once every 2 days or once every three days. In some embodiments, the dosing amount of the first administration can include a designated concentration of oxybutynin, and the dosing amount of the subsequent administration can include a different concentration of oxybutynin, e.g., a greater concentration of oxybutynin or a reduced concentration of oxybutynin.

In some embodiments, the disclosure provides a method of treating hot flashes in a human, the method comprising applying the composition as a spray to the skin of the human. In some embodiments, the composition is applied once within a 24 hour period. In some embodiments, the composition is application more than once within a 24 hour period. In some embodiments, the method comprises applying a composition comprising about 0.5% w/v to about 15% w/v oxybutynin.

In some embodiments, the disclosure provides a method of treating hyperhidrosis, osmiodrosis, or combinations thereof in a human, the method comprising applying the composition as a spray to the skin of the human. In some embodiments, the composition is applied once within a 24 hour period. In some embodiments, the composition is application more than once within a 24 hour period. In some embodiments, the method comprises applying a composition comprising about 0.5% w/v to about 15% w/v oxybutynin.

In some embodiments, the disclosure provides a method of treating incontinence, bladder spasms, pain following bladder surgery, or combinations thereof in a human, the method comprising applying the composition as a spray to the skin of the human. In some embodiments, the composition is applied once within a 24 hour period. In some embodiments, the composition is application more than once within a 24 hour period. In some embodiments, the method comprises applying a composition comprising about 0.5% w/v to about 15% w/v oxybutynin.

In some embodiments, the disclosure provides a method of treating moderate to severe vasomotor symptoms caused by menopause in a human female, the method comprising applying the composition as a spray to the skin of the human female. In some embodiments, the composition is applied once within a 24 hour period. In some embodiments, the composition is application more than once within a 24 hour period. In some embodiments, the method comprises applying a composition comprising about 0.5% w/v to about 15% w/v oxybutynin.

In some embodiments, the disclosure provides a method of treating hot flashes in a human, the method comprising applying a first application of the composition to a first site on the skin of the human, and applying a subsequent application of the composition to the skin of the human at a timepoint about 24 hours after the first application. In some embodiments, the method comprises applying a composition comprising about 0.5% w/v to about 15% w/v oxybutynin. In some embodiments, the subsequent application of the composition is applied at a site on the skin that is different from the first site. In some embodiments, the subsequent application of the composition is applied at the first site on the skin.

In some embodiments, the disclosure provides a method of treating hyperhidrosis, osmiodrosis, or combinations thereof in a human, the method comprising applying a first application of the composition to a first site on the skin of the human, and applying a subsequent application of the composition to the skin of the human at a timepoint about 24 hours after the first application. In some embodiments, the method comprises applying a composition comprising about 0.5% w/v to about 15% w/v oxybutynin. In some embodiments, the subsequent application of the composition is applied at a site on the skin that is different from the first site. In some embodiments, the subsequent application of the composition is applied at the first site on the skin.

In some embodiments, the disclosure provides a method of treating incontinence, bladder spasms, pain following bladder surgery, or combinations thereof in humans, the method comprising applying a first application of the composition to a first site on the skin of the human, and applying a subsequent application of the composition to the skin of the human at a timepoint about 24 hours after the first application. In some embodiments, the method comprises applying a composition comprising about 0.5% w/v to about 15% w/v oxybutynin. In some embodiments, the subsequent application of the composition is applied at a site on the skin that is different from the first site. In some embodiments, the subsequent application of the composition is applied at the first site on the skin.

In some embodiments, the disclosure provides a method of treating moderate to severe vasomotor symptoms caused by menopause in a human female, the method comprising applying a first application of the composition to a first site on the skin of the human, and applying a subsequent application of the composition to the skin of the human at a timepoint about 24 hours after the first application. In some embodiments, the subsequent application of the composition is applied at a site on the skin that is different from the first site. In some embodiments, the subsequent application of the composition is applied at the first site on the skin once the protective film from the previous application has been washed off.

In some embodiments, the composition forms a barrier film less than three minutes after application of the composition to the skin. In some embodiments, the composition forms a barrier film less than two minutes after application of the composition to the skin. In some embodiments, the composition forms a barrier film less than one minute after application of the composition to the skin. In some embodiments, the barrier film is capable of preventing transfer of oxybutynin to others. In some embodiments, the barrier film formed is water washable. In some embodiments, the barrier film formed from the first application of the composition is removed from the first site before the composition is applied to the first site in a subsequent application.

In some embodiments, the methods of treatment comprise applying a volume of about 50 μL to about 300 μL of the composition to the skin in a single actuation. In some embodiments, the methods of treatment comprises applying a maximum of 500 μL to the skin in a single actuation. In some embodiments, the methods of treatment comprise applying a maximum of 250 μL of the composition to the skin in a single actuation. In some embodiments, the methods of treatment comprise applying a maximum of 100 μL of the composition to the skin in a single actuation.

In some embodiments, the method comprises treating a human who is a breast cancer patient or a breast cancer survivor. In some embodiments, the method comprises treating a human who suffers from hyperhidrosis. In some embodiments, the method comprises treating a human who suffers from bladder spasms and pain.

In some embodiments, the composition comprises oxybutynin, octisalate, methacrylic acid and methyl methacrylate copolymer 1:2, and polyethylene glycol 400. In some embodiments, the composition comprises about 0.5% w/v to about 10% w/v oxybutynin. In some embodiments, the composition comprises about 1% w/v to about 10% w/v octisalate. In some embodiments, the composition comprises about 1% w/v to about 10% w/v methacrylic acid and methyl methacrylate copolymer 1:2. In some embodiments, the composition further comprises ethanol and tromethamine. In some embodiments, the composition comprises about 3% w/v to about 7% w/v octisalate, and about 3% w/v to about 7% w/v methacrylic acid and methyl methacrylate copolymer 1:2.

In some embodiments, the composition has a viscosity of less than 30 cPs at room temperature. In some embodiments, the composition has a viscosity of about 5 cPs to about 15 cPs.

In some embodiments, the present disclosure provides a spray container comprising the composition, and a metering valve. In some embodiments, the spray container further comprises a dose indicator.

### BRIEF DESCRIPTION OF THE FIGURES

FIG. 1 is a graph illustrating the skin permeation profiles of a 4% and a 6% oxybutynin film forming composition as outlined in Example 2.

FIG. 2 is a graph illustrating the results of a film breathability study performed using an occluded and a non-occluded oxybutynin film forming composition as outlined in Example 3.

### DETAILED DESCRIPTION OF THE INVENTION

The present disclosure relates to compositions comprising oxybutynin and methods suitable for the treatment of hot flashes, hyperhidrosis, osmiodrosis, incontinence, bladder spasms, pain following bladder surgery, vasomotor symptoms caused by menopause, and combinations thereof. In some embodiments, the disclosure provides a topical composition comprising oxybutynin, e.g., a topical spray. Oxybutynin compositions have been used previously in the forms of transdermal patches, sprays and gels. However, the present disclosure provides for a new composition which forms a film on the skin after the spray has dried. The film acts as barrier that prevents transfer of oxybutynin from the application site to others, e.g., the user's partner. Additionally, the film is washable as it can conveniently be removed with water while performing daily activities such as a shower. In some embodiments, the sprayable compositions described herein can be sprayed directly on the skin surface, and do not need to be spread over the skin by touching with fingers or with a separate applicator. In some embodiments, drug absorption into the skin is rapid. In some embodiments drug absorption from the spray into the skin is complete when the protective film has dried (<5 mins). In some embodiments, the film can be washed off once it has dried. During solvent evaporation, the excipients included in the spray prevent recrystallization of oxybutynin, a feature that is important for its penetration of the skin. In some embodiments, the oxybutynin in the composition described herein does not crystallize when the composition is applied to the skin. In some embodiments, the protective film is breathable which prevents erythema of the skin developing over long-term use. In some embodiments, direct application to the skin via a spray provides for increased convenience, and lower transmission of the oxybutynin to the hands/fingers or other body parts for which it is not needed. In some embodiments, the sprayable compositions described herein can be applied quickly. In some embodiments, the sprayable compositions described herein can be applied quickly over a large area of the skin.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. A used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.,” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, “room temperature” is an indoor temperature suitable for long term storage of biological matter and laboratory experimentation, typically ranging between 15-28° C. In embodiments, room temperature is from 20-25° C.

The present disclosure is directed to a composition comprising about 1% w/v to about 20% w/v of oxybutynin, a penetration enhancer, at least 65% w/v of an aliphatic solvent and a film forming excipient, wherein the film forming excipient has a solubility in water, at a pH between 1 and 10, and wherein the composition is a sprayable liquid solution that forms a washable film when sprayed on skin.

The present disclosure is also directed to a composition comprising about 1% w/v to about 8% w/v of oxybutynin, a penetration enhancer, at least 65% w/v of an aliphatic solvent and a film forming excipient, wherein the film forming excipient has a solubility in water, at a pH between 1 and 10, and wherein the composition is a sprayable liquid solution that forms a washable film when sprayed on skin.

The present disclosure is also directed to a composition comprising about 8% w/v of oxybutynin, a penetration enhancer, about 35% to about 45% w/v of an aliphatic solvent and a film forming excipient, wherein the film forming excipient has a solubility in water, at a pH between 1 and 10, and wherein the composition is a sprayable liquid solution that forms a washable film when sprayed on skin.

The present disclosure is also directed to a composition comprising about 6% w/v of oxybutynin, a penetration enhancer, about 35% to about 45% w/v of an aliphatic solvent and a film forming excipient, wherein the film forming excipient has a solubility in water, at a pH between 1 and 10, and wherein the composition is a sprayable liquid solution that forms a washable film when sprayed on skin.

Various concentrations of oxybutynin can be used in the composition. In some embodiments, the composition comprises about 1% w/v to about 20% w/v oxybutynin. In some embodiments, the composition comprises about 1% w/v to about 10% w/v oxybutynin. In some embodiments, the composition comprises about 1% w/v to about 15% w/v oxybutynin. In some embodiments, the composition comprises about 1% w/v to about 16% w/v oxybutynin. In some embodiments, the composition comprises about 1% w/v to about 8% w/v oxybutynin. In some embodiments, the composition comprises about 1% w/v to about 7% w/v oxybutynin. In some embodiments, the composition comprises about 1% w/v to about 6% w/v oxybutynin. In some embodiments, the composition comprises about 1% w/v to about 5% w/v oxybutynin. In some embodiments, the composition comprises about 1% w/v to about 4% w/v oxybutynin. In some embodiments, the composition comprises about 1% w/v to about 3% w/v oxybutynin. In some embodiments, the composition comprises about 1% w/v to about 2% w/v oxybutynin. In some embodiments, the composition comprises about 2% w/v to about 8% w/v oxybutynin. In some embodiments, the composition comprises about 2% w/v to about 7% w/v oxybutynin. In some embodiments, the composition comprises about 2% w/v to about 6% w/v oxybutynin. In some embodiments, the composition comprises about 2% w/v to about 5% w/v oxybutynin. In some embodiments, the composition comprises about 2% w/v to about 4% w/v oxybutynin. In some embodiments, the composition comprises about 2% w/v to about 3% w/v oxybutynin. In some embodiments, the composition comprises about 3% w/v to about 8% w/v oxybutynin. In some embodiments, the composition comprises about 3% w/v to about 7% w/v oxybutynin. In some embodiments, the composition comprises about 3% w/v to about 6% w/v oxybutynin. In some embodiments, the composition comprises about 3% w/v to about 5% w/v oxybutynin. In some embodiments, the composition comprises about 3% w/v to about 4% w/v oxybutynin.

In some embodiments, the composition comprises about 4% w/v to about 8% w/v oxybutynin. In some embodiments, the composition comprises about 4% w/v to about 7% w/v oxybutynin. In some embodiments, the composition comprises about 4% w/v to about 6% w/v oxybutynin. In some embodiments, the composition comprises about 4% w/v to about 5% w/v oxybutynin. In some embodiments, the composition comprises about 5% w/v to about 8% w/v oxybutynin. In some embodiments, the composition comprises about 5% w/v to about 7% w/v oxybutynin. In some embodiments, the composition comprises about 5% w/v to about 6% w/v oxybutynin. In some embodiments, the composition comprises about 6% w/v to about 8% w/v oxybutynin. In some embodiments, the composition comprises about 6% w/v to about 7% w/v oxybutynin. In some embodiments, the composition comprises about 7% w/v to about 8% w/v oxybutynin.

In some embodiments, the composition comprises about 1% w/v oxybutynin. In some embodiments, the composition comprises about 2% w/v oxybutynin. In some embodiments, the composition comprises about 3% w/v oxybutynin. In some embodiments, the composition comprises about 4% w/v oxybutynin. In some embodiments, the composition comprises about 5% w/v oxybutynin. In some embodiments, the composition comprises about 6% w/v oxybutynin. In some embodiments, the composition comprises about 7% w/v oxybutynin. In some embodiments, the composition comprises about 8% w/v oxybutynin. In some embodiments, the composition comprises about 9% w/v oxybutynin. In some embodiments, the composition comprises about 10% w/v oxybutynin. In some embodiments, the composition comprises about 11% w/v oxybutynin. In some embodiments, the composition comprises about 12% w/v oxybutynin. In some embodiments, the composition comprises about 13% w/v oxybutynin. In some embodiments, the composition comprises about 14% w/v oxybutynin. In some embodiments, the composition comprises about 15% w/v oxybutynin. In some embodiments, the composition comprises about 16% w/v oxybutynin. In some embodiments, the composition comprises about 17% w/v oxybutynin. In some embodiments, the composition comprises about 18% w/v oxybutynin. In some embodiments, the composition comprises about 19% w/v oxybutynin. In some embodiments, the composition comprises about 20% w/v oxybutynin.

In some embodiments, the topical composition can comprise a penetration enhancer. The term “penetration enhancer” refers to any compound or composition that penetrate into the skin and interact with skin constituents to promote drug flux or reversibly decrease the barrier resistance. Penetration enhancers can include substances which promote the dermal absorption of oxybutynin through the skin temporarily by transiently disturbing the lipid bilayer structure in the stratum corneum barrier or enhancing the solubility of the drug in the skin to facilitate drug delivery. In some embodiments, the penetration enhancer can include any penetration enhancer known to be pharmaceutically acceptable. In some embodiments, the penetration enhancer can include any skin enhancer as found in the U.S. Pharmacopeia, or otherwise known in the art, e.g., M. E. Lane, Skin Permeation Enhancers, Int. J. Pharma 447(1-2):12-21 (2013). In some embodiments, the penetration enhancer can include 1-dodecylazacycloheptan-2-one, isopropyl myristate, octisalate, oleic acid, diethylene glycol monoethyl ether (Transcutol®), or combination thereof. In some embodiments, one, two, three or greater than three penetration enhancers are on the topical composition. In some embodiments, two penetration enhancers are in the composition.

Various concentrations of penetration enhancers can be present in the topical composition. The permeation enhancer can be adjusted to facilitate the penetration of oxybutynin. In some embodiments, the composition comprises about 0.1% w/v to about 20% w/v, from about 0.1% w/v to about 15% w/v, from about 0.1% w/v to about 10% w/v, about 0.1% w/v to about 5% w/v, or about 0.5% w/v to about 8% w/v penetration enhancer. In some embodiments, the composition comprises about 4% to about 25% by weight penetration enhancer. In some embodiments, the composition comprises about 4% to about 20% by weight penetration enhancer. In some embodiments, the composition comprises about 4% to about 15% by weight penetration enhancer. In some embodiments, the composition comprises about 4% to about 10% by weight penetration enhancer. In some embodiments, the composition comprises about 10% to about 15% by weight penetration enhancer. In some embodiments, the composition comprises about 10% to about 20% by weight penetration enhancer. In some embodiments, the composition comprises about 15% to about 20% by weight penetration enhancer. In some embodiments, the composition comprises about 15% to about 25% by weight penetration enhancer.

Various aliphatic solvents can be used in the composition of the present disclosure. The term “aliphatic solvent” enhancer” refers to any solvent comprising compounds that are without a ring structure, e.g., without an aromatic ring structure. In some embodiments, the solvent is suitable for solubilizing oxybutynin, e.g., a non-aromatic solvent, which is pharmaceutically acceptable. In some embodiments, the solvent is an alcoholic aliphatic solvent. In some embodiments, the aliphatic solvent is ethylene, isooctane, acetylene, propene, propane, squalene, acetone, ethanol, methanol, propanol, butanol, isopropyl alcohol and polyethylene. In some embodiments, aliphatic solvents can include more than one solvent, e.g., a mixture of aliphatic solvents. In some embodiments, the solvent is ethanol or a mixture of ethanol with other aliphatic solvents. In some embodiments, the solvent comprises acetone, ethanol, and/or isopropyl alcohol. In some embodiments, the aliphatic solvent has a viscosity suitable for administering via an aerosol spray or a mist.

In some embodiments, the aliphatic solvent comprises two solvents at a ratio of about 1:100 to about 100:1. In some embodiments, the aliphatic solvent comprises two solvents at a ratio of about 5:95 to about 95:5. In some embodiments, the aliphatic solvent comprises two solvents at a ratio of about 20:80 to about 80:20. In some embodiments, the aliphatic solvent comprises two solvents at ratio of about 30:70 to about 70:30. In some embodiments, the aliphatic solvent comprises two solvents at a ratio of about 40:60 to about 60:40. In some embodiments, the aliphatic solvent comprises two solvents at a ratio of about 50:50.

In some embodiments, the aliphatic solvent comprises three solvents at a ratio of about 1:1:1 to about 1:1:10. In some embodiments, the aliphatic solvent comprises three solvents at a ratio of about 1:2:2 to about 1:2:4. In some embodiments, the aliphatic solvent comprises three solvents at a ratio of about 1:3:1 to about 1:3:6. In some embodiments, the aliphatic solvent comprises three solvents at a ratio of about 1:4:1 to about 1:4:12.

The total aliphatic solvent concentration can be around 20% to about 95% weight of the composition, but in some embodiments the aliphatic solvent is in a sufficient quantity to dissolve the other excipients and oxybutynin. In some embodiments, the composition comprises about 65% weight aliphatic solvent. In some embodiments, the composition comprises about 10% to about 80% by weight aliphatic solvent. In some embodiments, the composition comprises about 20% to about 80%, about 30% to about 80%, about 40% to about 80% or about 50% to about 80% by weight aliphatic solvent. In some embodiments, the composition comprises about 52% to about 68%, about 54% to about 66%, about 56% to about 64% or about 58% to about 62% by weight aliphatic solvent. In some embodiments, the composition comprises about 35% to about 45% by weight aliphatic solvent. In some embodiments, the composition comprises about 60% by weight aliphatic solvent. In some embodiments, the composition comprises less than 80%, less than 70%, less than 65%, less than 62% or less than 61% by weight of aliphatic solvent. In some embodiments, the composition comprises about 70% to about 85% alcoholic solvent, about 72% to about 82% aliphatic solvent, or about 74% to about 80% aliphatic solvent by weight. In some embodiments, the composition comprises about 75%, about 76%, about 77%, about 78%, about 79%, about 80%, about 81%, about 82% or about 83% aliphatic solvent by weight. In some embodiments, the reduced aliphatic solvent concentration can result in reduced adverse effect, e.g., inflammation or irritation.

In some embodiments, the aliphatic solvent is a volatile solvent. For example, in some embodiments, the solvent evaporates, i.e., 500 μL, 250 μL, or 100 μL vaporizes in less than two minutes, in less than one minute or in less than 30 seconds after the composition is sprayed onto a surface at room temperature and 1 atmospheric pressure.

The present disclosure provides for oxybutynin compositions comprising a film forming excipient. The disclosure provides for compositions, e.g., topical spray compositions comprising a film forming excipient, wherein the film forming excipient comprises a polyacrylate polymer, polyvinyl polymer or a cellulose polymer. In some embodiments, such compositions provide for uniform drug distribution and dose, increased bioavailability, continuous drug release and longer-lasting effect to the treated area, while minimizing transfer of oxybutynin. In some embodiments, the long-lasting effect of the present compositions allow for a reduced number of total administrations of the oxybutynin composition. In some embodiments, the long-lasting effect of the present compositions allow for a reduced frequency of administration of the oxybutynin composition.

The film forming excipient is an excipient, preferably a polymer, that is soluble in aliphatic solvents, preferably in ethanol or a mixture of ethanol with other solvents. The film forming excipient is also soluble in aqueous solutions, preferably water. Although, for film forming excipients having pH dependent solubility, the pH of the aqueous solution must be above or below the specific trigger pH for that excipient to dissolve. For example, some of these film forming excipients dissolve in aqueous solutions only above pH 6.0, only above pH 7.0, or only below pH 5.0. In some embodiments, the film forming excipient has a solubility in water, at a pH between 1 and 10.

In some embodiments, the composition comprises a film forming excipient comprising a polyacrylate polymer, polyvinyl polymer or a cellulose polymer. Polyacrylate polymers are commercially available and known to those in the art, and can refer to a group of polymers prepared from acrylate monomers. Polyacrylate polymers can include methacrylic acid and methyl methacrylate copolymer 1:1, methacrylic acid and methyl methacrylate copolymer 1:2, and poly(butylmethacrylate-co-(2-dimethylaminoethyl)methacrylate-co-methyl methacrylate 1:2:1. The term cellulose polymer refers to a group of polymers prepared from glucose monomers. Polyvinyl polymers are commercially available and known to those in the art, and can refer to a group of polymers prepared from vinyl monomers. In some embodiments, the film forming excipients used herein can exhibit properties that are important for topical compositions including high tensile strength, lack of deformity under minimal tensile stress, and low sensitivity to microbial contamination. In some embodiments, the film forming excipient allows for penetration of oxybutynin into the skin. In some embodiments, the film forming excipient allows moisture vapor to pass through the skin.

In some embodiments, the film forming excipient is a polyacrylate polymer. In some embodiments, the polyacrylate polymer is methacrylic acid and methyl methacrylate copolymer. In some embodiments, the methacrylic acid and methacrylate copolymer are in a ratio of about 1:4 to about 4:1, about 1:3 to about 3:1, about 1:2 to about 2:1. In some embodiments, the methacrylic acid and methacrylate copolymer are in ratio of 1:1. In some embodiments, the methacrylic acid and methacrylate copolymer are in ratio of 1:2. In some embodiments the polyacrylate polymer is methacrylic acid and methyl methacrylate copolymer 1:2. In some embodiments the polyacrylate polymer is poly(butylmethacrylate-co-(2-dimethylaminoethyl)methacrylate-co-methyl methacrylate 1:2:1. Polyvinyl polymers can include polyvinyl alcohol. Various cellulose polymers are known in the art, and can include hypromellose, hydroxypropyl cellulose, and ethyl cellulose

In some embodiments, the cellulose polymer is hypromellose. In some embodiments the cellulose polymer is hydroxypropyl cellulose. In some embodiments the cellulose polymer is ethyl cellulose. In some embodiments, the topical composition can have various combinations of penetration enhancers and film forming excipients. In some embodiments, the composition comprises the penetration enhancer octisalate, and the film forming excipient methacrylic acid and methyl methacrylate copolymer 1:1.

In some embodiments, the topical composition can have more than one film forming excipient. In some embodiments, the composition comprises about 4% w/v to about 7% w/v film forming excipient. In some embodiments, the composition comprises about 4% w/v to about 6% w/v film forming excipient. In some embodiments, the composition comprises about 4% w/v to about 5% w/v film forming excipient. In some embodiments, the composition comprises about 5% w/v to about 7% w/v film forming excipient. In some embodiments, the composition comprises about 5% w/v to about 7% w/v film forming excipient. In some embodiments, the composition comprises about 5% w/v to about 7% w/v film forming excipient. In some embodiments, the composition comprises about 6% w/v to about 7% w/v film forming excipient.

In some embodiments, the composition forms a washable film. In some embodiments, the present disclosure provides topical compositions that can be washed off via convenient methods, i.e., showering, when absorption of the drug into the skin is complete. In some embodiments, washability includes removal of the film without the use of a detergent. The ability to easily wash off the dried film in the composition allows the user to reapply the subsequent dose of the composition at the previous site of application. A washability enhancer can be included in the composition to make the film more washable, i.e., easier to remove from skin with water or soap and water, particularly when the film forming excipient is an excipient that requires a certain pH to dissolve in water or an aqueous environment, such as methacrylic acid-methyl methacrylate copolymer (1:2) (Eudragit® S100). In some cases, the washability enhancer also acts as a plasticizer. Moreover, the film forming excipient, or mixture of film forming excipient with a plasticizer and/or washability enhancer, prevents recrystallization of oxybutynin and maintains oxybutynin in an amorphous state during and after solvent evaporation. In some embodiments, the washability enhancer is a low molecular weight polyethylene glycol (PEG) containing ethylene glycol polymer of various molecular weights, such as polyethylene glycol 300, 400 or 600.

The concentration of washability enhancer in the composition can be about 1% to about 300% of the weight of the film forming excipient in the composition. In some embodiments it is about 50% to about 250% of the weight of the film forming excipient in the composition. In other embodiments, it is about 20% to about 100% of the weight of the film forming excipient in the composition. It can also be is about 40%, about 45%, about 50%, about 55%, about 60%, about 70%, about 80%, about 90%, about 100%, about 120%, about 140%, about 160%, about 180%, about 190%, about 200%, about 210%, about 220%, about 230%, about 240%, or about 250% of the weight of the film forming excipient in the composition. With regard to the total weight of the composition, in some embodiments, the concentration of washability enhancer in the composition can be about 0.1% w/v to about 20% w/v, about 0.1% w/v to about 15% w/v, about 0.1% w/v to about 10% w/v, about 0.1% w/v to about 5% w/v, about 0.5% w/v to about 5% w/v, or about 1% w/v to about 5% w/v of the composition.

The composition can include one or more plasticizers such as dibutyl sebacate, triethyl citrate, triacetin, glycerol, a low molecular weight polyethylene glycol, e.g., polyethylene glycol 300, 400 or 600, and/or propylene glycol. The concentration of plasticizer in the composition can be about 0.1% w/v to about 20% w/v, about 0.1% w/v to about 15% w/v, about 0.1% w/v to about 10% w/v, about 0.1% w/v to about 5% w/v, about 0.5% w/v to about 5% w/v, or about 1% w/v to about 5% w/v of the composition. In some embodiments it is about 0.5% w/v, about 1% w/v, about 1.5% w/v, about 2% w/v, about 2.5% w/v, about 3% w/v, about 3.5% w/v, or about 4% w/v, of the composition. In some embodiments, the plasticizer is also a washability enhancer.

The composition can also include one or more viscosity increasing agents, e.g., povidone, glycerin, hydroxypropyl cellulose, methylcellulose, and/or carboxymethylcellulose. The concentration of the viscosity increasing agent, if included, can be from about 0.1% w/v to about 10% w/v, about 0.1% w/v to about 8% w/v, about 0.1% w/v to about 5% w/v of the composition, e.g., about 0.5% w/v, about 1% w/v, about 1.5% w/v, about 2% w/v, or about 2.5% w/v of the composition. In some embodiments, the viscosity increasing agent is in an amount that allows the composition to still be a sprayable liquid solution. For example, in some embodiments, the viscosity of the composition is less than 40 mPas, less than 30 mPas, less than 20 mPas, less than 10 mPas or less than 5 mPas at room temperature and 1 atmospheric pressure.

In some embodiments, the topical compositions can comprise viscosity increasing agents. In some embodiments, the viscosity increasing agent is a bioadhesive. Such agents include Carbopol®. Carbopol® polymers are high molecular weight, crosslinked polyacrylic acid polymers. The polymers differ by crosslink density and can be grouped into the following categories: (1) Carbopol® homopolymers, such as acrylic acid crosslinked with allyl sucrose or allyl pentaerythritol, e.g., Carbopol® 71G NF (viscosity 4,000-11,000), 971P NF (viscosity 4,000-11,000), 974P NF, (viscosity 29,400-39,400), 980 NF (viscosity 40,000-60,000), 981 NF (viscosity 4000-10,000), 5984 EP (viscosity 30,500-39,400), 934 NF (viscosity 30,500-39,400), 934P NF (viscosity 29,400-39,400), 940 NF (viscosity 40,000-60,000), 941 NF (viscosity 4,000-10,000); and (2) Carbopol® copolymers: acrylic acid and C10-C30 alkyl acrylate crosslinked with allyl pentaerythritol, e.g., Carbopol® 1342 NF, viscosity 9,500-26,500.

The compositions of the present disclosure can also include other formulation excipients, added, e.g., to achieve a desired consistency or appearance, or to protect the formulation components from degradation and oxidation. Such excipients include, for example, cosolvents, stabilizing agents, antioxidants, humectants, preservatives, pH modifiers, colorant, dye, and fragrances known in the art of formulation.

Another optional excipient of the compositions is a pH modifier, such as tromethamine. In some embodiments, the concentration of the pH modifier can be about 0.05 g/L to about 0.2 g/L. Here, grams per liter (g/L) refers to grams of organic proton acceptor per liter aliphatic solvent in the composition. In some embodiments, the concentration of the pH modifier is about 0.05 g/L to about 0.1 g/L, about 0.1 g/L to about 0.15 g/L, or about 0.15 to about 0.2 g/L. In some embodiments of the aliphatic solvent comprises tromethamine.

In some embodiments, particularly for application to the skin, the composition can comprise a fragrance or perfume to impart a desired aroma, or to mask odors that can be associated with other components of the composition. In some embodiments, the concentration of the fragrance is about 0.01% w/v to about 5% w/v of the composition, or about 0.1% w/v to about 1% w/v of the composition.

Any fragrance suitable for application to the skin can be used herein including a wide variety of fragrances and perfumes that are known to those skilled in the art. In some embodiments, the amount of fragrance can be effective for providing a noticeable aroma to the composition, or for masking undesired aroma of the composition. In some embodiments, the fragrance does not impart excessive stinging to the skin, especially to broken or irritated skin.

Typical fragrances are described in Arctander, Perfume and Flavour Chemicals (Aroma Chemicals), Vol. I and II (1969, Allured Publishing Corporation, 1969 (ISBN 0931710375, 9780931710377), and Arctander, Perfume and Flavor Materials of Natural Origin (1994, by Allured Pub Corp) (ISBN 0931710367; ISBN13: 9780931710360). Fragrance used in the present composition can also contain solubilizers, diluents, or solvents which are well known in the art.

In some embodiments, the present disclosure provides a topical composition comprising oxybutynin, octisalate, methacrylic acid and methyl methacrylate copolymer 1:2, and polyethylene glycol 400. In some embodiments, the composition comprises about 0.5% w/v to about 10% w/v oxybutynin. In some embodiments, the composition comprises about 1% w/v to about 10% w/v octisalate. In some embodiments, the composition comprises about 1% w/v to about 10% w/v methacrylic acid and methyl methacrylate copolymer 1:2. In some embodiments, the composition comprises the composition comprises about 3% w/v to about 7% w/v octisalate, and about 3% w/v to about 7% w/v methacrylic acid and methyl methacrylate copolymer 1:2. In some embodiments, the composition further comprises ethanol and tromethamine.

In some embodiments, topical application of oxybutynin without a protective cover or barrier over the area can leave unabsorbed drug exposed, which creates a risk of transfer of oxybutynin to other persons or surfaces and unwanted side effects. Additionally, in some embodiments, the loss of oxybutynin after transfer from the user's skin to other surfaces can also alter the amount of the drug remaining on the skin necessary for its intended therapeutic effect. The present disclosure provides a topical composition that prevents transfer of oxybutynin.

In some embodiments, the barrier film is occlusive. In some embodiments, the occlusive film is transparent. In some embodiments, occlusive refers to a film that forms a protective layer on a surface, meaning the film prevents transference of oxybutynin to other humans. In some embodiments, the occlusive film forms a protective layer against bacteria and viruses. In some embodiments, the occlusive film is breathable and allows moisture vapor to be able to pass through the film, which maintains the integrity of the skin.

As the barrier film prevents transfer of oxybutynin, it is important for the topical composition to quickly dry after application and form the barrier film. In some embodiments, the topical composition dries quickly after application. For example, in some embodiments, the topical composition dries within 90 seconds, within 80 seconds, within 70 seconds or within 60 seconds after application. In some embodiments, the topical composition forms a barrier film less than three minutes after application to the skin. In some embodiments, the topical composition forms a barrier film less than two minutes after application to the skin. In some embodiments, the topical composition forms a barrier film in less than one minute after application to the skin. For example, in some embodiments, the topical composition forms a barrier film within 90 seconds, within 80 seconds, within 70 seconds or within 60 seconds after application.

In some embodiments, the topical composition dries quickly after application. For example, in some embodiments, the topical composition dries within 90 seconds, within 80 seconds, within 70 seconds or within 60 seconds after application of 500 μL, 250 μL, 100 μL or 50 μL of the composition to the skin at room temperature and 1 atmospheric pressure. In some embodiments, the topical composition forms a barrier film less than three minutes after application of 500 μL, 250 μL, 100 μL or 50 μL of the composition to the skin at room temperature and 1 atmospheric pressure. In some embodiments, the topical composition forms a barrier film less than two minutes after application of 500 μL, 250 μL, 100 μL or 50 μL of the composition to the skin at room temperature and 1 atmospheric pressure. In some embodiments, the topical composition forms a barrier film in less than one minute after application of 500 μL, 250 μL, 100 μL or 50 μL of the composition to the skin at room temperature and 1 atmospheric pressure. For example, in some embodiments, the topical composition forms a barrier film within 90 seconds, within 80 seconds, within 70 seconds or within 60 seconds after application of 500 μL, 250 μL, 100 μL or 50 μL of the composition at room temperature and 1 atmospheric pressure.

In some embodiments, the use of transdermal patches or topical solutions with a protective cover or barrier over the area can result in skin sensitization and/or irritation, discomfort from adhesives, and imperfect skin adhesion. The present disclosure provides compositions that are designed to remain intact for an extended period of time, e.g., for 24 hours without the need to cover or otherwise protect the composition. In some embodiments, it is important for moisture vapor to be able to pass through the film during the time the film is present in order to ensure that the underlying skin is able to breathe and allow sweat to evaporate rather than block skin pores. In some embodiments, the present disclosure provides compositions that have a water vapor transmission rate, as a fraction compared to a non-occluded control, not less than 0.50 over a 48 hour period, not less than 1.0 over a 48 hour period, not less than 1.2 over a 48 hour period the composition, when dried on a porous substrate. In some embodiments, the barrier film of the composition prevents reactions of the skin that are associated with non-breathable topical and transdermal treatments such as intense itching, redness (erythema), and blistering.

High viscosity topical oxybutynin formulations, e.g., creams, lotions and gels, require application to the skin with the palm or fingers of the hand of the user, and thus these types of formulations are associated with a significant risk for secondary oxybutynin exposure. The present claims feature a “sprayable liquid solution” which can be applied directly to the skin in an amount effective in delivering the required dosage needed for the treatment of conditions caused by vasomotor symptoms. In some embodiments, the “sprayable liquid solution” has low viscosity (about 15 cPs to about 45 cPs) and can be administered in smaller volumes, i.e., in a volume less than 500 μL per actuation. In some embodiments, the topical composition has a viscosity suitable for administering via an aerosol spray and/or a mist. For example, in some embodiments, the topical composition has a viscosity of less than 200 cPs, less than 100 cPs, less than 50 cPs or less than 30 cPs at room temperature. In some embodiments, the topical formation has a viscosity of about 1 cp to about 10 cPs, about 5 cPS to about 20 cPs or about 20 cPs to about 30 cPs at room temperature. In some embodiments, the topical composition has a viscosity of about 5 cPs to about 15 cPs. As used throughout the present disclosure, viscosity is measured at standard conditions, e.g., 1 atm pressure and 25° C. temperature.

The topical composition of the present disclosure can be administered by means to known in the art. In some embodiments, the disclosure provides a spray container for administering the composition. In some embodiments, the disclosure provides a spray container comprising the topical compositions as described herein. In some embodiments, the spray container is a sealed and pressurized device, e.g., an aerosol container. In some embodiments, the spray container is a sealed and pressurized device, an aerosol container, with a means for providing an aerosol spray of the topical composition, e.g., an aerosol nozzle. In some embodiments, the spray container further comprises a dose indicator. In some embodiments, the spray container comprises an amount of the topical composition sufficient for a single administration of the composition. In some embodiments, the spray container comprises an amount of the topical composition sufficient for two administrations of the composition. In some embodiments, the spray container comprises an amount of the topical composition sufficient for three or more administrations of the composition.

In embodiments in which the spray container is a pressurized device, the pressure in the sealed and pressurized device can be any pressure suitable for delivery of the topical composition. In some embodiments, the pressure is about 28 psi to about 145 psi at 25° C.

In some embodiments, the spray container comprising the topical composition is a misting device, with a means for providing a mist of the topical composition. As used herein, the misting device would include any device which is capable of producing fine mist particles of the topical composition. In some embodiments, the misting device produces mist particles of the topical composition with an average diameter of about 20 μm to about 150 μm.

### Methods of Treatment

In some embodiments, the disclosure provides a method of treating hot flashes in a human female, the method comprising applying a first application of any of the compositions described herein to a first site on the skin of the human female and applying a subsequent application of the composition to the skin of the human at a timepoint about 24 hours after the first application. In some embodiments, the human is a human female. In some embodiments, the human is a human male, e.g., a male who suffers from prostate cancer who suffers from hot flashes. As used herein, the phrase “a timepoint about 24 hours after the first application” refers to a subsequent administration, that is about 1 day later, e.g., 24 hours±6 hours, ±4 hours, ±2 hours, etc. In some embodiments, the phrase “a timepoint about 24 hours after the first application” refers to a subsequent administration about one day after the initial application, e.g., if the initial application was administered in the morning, the subsequent administration could occur in the following morning, etc.

In some embodiments, the disclosure provides a method of treating hyperhidrosis, osmiodrosis, or combinations thereof in humans, the method comprising applying a first application of any of the compositions described herein to a first site on the skin of the human and applying a subsequent application of the composition to the skin of the human at a timepoint about 24 hours after the first application.

In some embodiments, the disclosure provides a method of treating incontinence, bladder spasms, pain following bladder surgery, or combinations thereof in humans, the method comprising applying a first application of any of the compositions described herein to a first site on the skin of the human, and applying a subsequent application of the composition to the skin of the human at a timepoint about 24 hours after the first application. In some embodiments, the spray may be applied more than once within a 24-hour period.

In some embodiments, the disclosure provides a method of treating moderate to severe vasomotor symptoms caused by menopause in a human female, the method comprising applying a first application of any of the compositions described herein to a first site on the skin of the human, and applying a subsequent application of the composition to the skin of the human at a timepoint about 24 hours after the first application.

The term “subsequent” as used herein refers to applications of the topical compositions that follow or come after another application in time, order, or place. For example, an application of the topical composition that is 24 hours after the first application of the composition to the skin is a subsequent application.

In some embodiments, the subsequent application is applied one hour to one week after the first application. In some embodiments, the subsequent application is applied two hours to 4 days after the first application. In some embodiments, the subsequent application is applied four hours to two days after the first application. In some embodiments, the subsequent application is applied six hours to one day after the first application. In some embodiments, the subsequent application is applied twelve hours to one day after the first application. In some embodiments, the subsequent application is applied “as needed” or upon the occurrence of a symptom associated with menopause.

The topical compositions described herein can be used in the treatment of vasomotor symptoms cause by menopause in a human female. Vasomotor symptoms caused by menopause are characterized by hot flashes accompanied by sweating and flushing, and experienced predominantly around the head, neck, chest, and upper back of the human body. Women afflicted with breast cancer have been reported to be at higher risk of hot flashes for longer-term with more severe hot flashes due to chemotherapy-induced menopause and treatment with antiestrogens. In some instances, for patients with breast cancer, hot flashes can interfere with adjuvant therapy compliance and can cause increased pain. In some instances, women with breast cancer cannot be prescribed hormone replacement therapy, which is currently the most effective treatment for hot flashes. The only approved non-hormonal treatment for hot flashes, paroxetine, interferes with breast cancer medications such as tamoxifen and aromatase inhibitors. Oxybutynin does not interfere with these cancer treatments. Given orally, oxybutynin has been shown to reduce hot flashes by 75% (Leon-Ferre et al., 2020). The present disclosure provides a topical spray formulation of oxybutynin that can provide fewer adverse events and can be well suited for breast cancer patients who currently have few options regarding the management of their more severe hot flashes.

In some embodiments, the topical compositions are used to treat hot flashes, in a human. In some embodiments, the topical compositions are used to treat hyperhidrosis, osmiodrosis, or combinations thereof in a human. In some embodiments, the disclosure provides a method of treating hot flashes, hyperhidrosis, osmiodrosis, or combinations thereof, the method comprising applying the compositions described herein as a spray to the skin of a human suffering from vasomotor symptoms. In some embodiments, the method of treating hot flashes, hyperhidrosis, osmiodrosis, or combinations thereof comprises applying the composition described herein once within a 24 hour period. In some embodiments, the method of treating hot flashes comprises applying a first application of the composition on the skin of the human, and applying a subsequent application of the composition to the skin of the human at a timepoint about 24 hours after the first application. In some embodiments, the human receiving treatment for hot flashes, hyperhidrosis, osmiodrosis, or combinations thereof is a cancer patient, e.g., a breast cancer patient. In some embodiments, the method of treating hyperhidrosis, osmiodrosis, or combinations thereof comprises applying a first application of the composition on the skin of the human, and applying a subsequent application of the composition to the skin of the human at a timepoint about 24 hours after the first application. In some embodiments, the human receiving treatment for hot flashes, hyperhidrosis, osmiodrosis, or combinations thereof is a cancer patient, e.g., a male prostate cancer patient suffering from hot flashes, hyperhidrosis, osmiodrosis, or combinations thereof.

The topical compositions described herein can be used in the treatment of urinary conditions including incontinence, bladder spasms, pain following bladder surgery, or combinations thereof in a human. In some embodiments, the method of treating incontinence, bladder spasms, pain following bladder surgery, or combinations thereof comprises applying the composition once within a 24 hour period. In some embodiments, the method of treating incontinence, bladder spasms, pain following bladder surgery, or combinations thereof comprises applying a first application of the composition on the skin of the human, e.g., female human, and applying a subsequent application of the composition to the skin of the human at a timepoint about 24 hours after the first application. In some embodiments, the human receiving treatment for incontinence, bladder spasms, pain following bladder surgery, or combinations thereof is a cancer patient, e.g., a breast cancer patient.

In some embodiments, the human, e.g., human female, receiving treatment for hot flashes, hyperhidrosis, osmiodrosis, or combinations thereof also suffers from incontinence, bladder spasms, pain following bladder surgery, or combinations thereof. In some embodiments, the human receiving treatment for incontinence, bladder spasms, pain following bladder surgery, or combinations thereof also suffers from hot flashes, hyperhidrosis, osmiodrosis, or combinations thereof.

The term “treatment” as used herein refers to reducing the severity of a symptom associated with hot flashes, hyperhidrosis, osmiodrosis, incontinence, bladder spasms, pain following bladder surgery, or combinations thereof. Thus, in some embodiments, methods provided herein provide palliative care for hot flashes, hyperhidrosis, osmiodrosis, incontinence, bladder spasms, pain following bladder surgery, or combinations thereof. In some embodiments, the term “treatment” refers to eliminating a symptom associated with hot flashes, hyperhidrosis, osmiodrosis, incontinence, bladder spasms, pain following bladder surgery, or combinations thereof. Thus, in some embodiments, the methods provided herein provide curative care associated with on hot flashes, hyperhidrosis, osmiodrosis, incontinence, bladder spasms, pain following bladder surgery, or combinations thereof. In some embodiments, the methods described herein reduce and/or eliminate the severity one, two, three or more than three symptoms associated with hot flashes, hyperhidrosis, osmiodrosis, incontinence, bladder spasms, pain following bladder surgery, or combinations thereof.

To achieve a dosage amount sufficient for the intended therapeutic effect, it is often necessary to apply large amounts of oxybutynin formulations to large surface areas of skin. However, administration of large volumes of a drug increases the likelihood that a solution does not dry in time, and then drips in runoff from the application site, which can result in an inconsistent and inaccurate amounts delivered. Additionally, drug runoff increases the risk of transfer to other persons and unwanted side effects. Achieving ideal dosing concentrations and volumes is critical to achieving consistent and accurate dosage amount in a patient and preventing transfer to others. In some embodiments, the present disclosure provides a composition for which the required volume of composition administered is reduced, and provides a film forming layer which reduces transference.

The topical compositions described herein can be applied in a volume of about 500 μL or less in a single actuation. In some embodiments, the composition is applied in a volume of about 300 μL or less in a single actuation. In some embodiments, the composition is applied in a volume of about 250 μL or less in a single actuation. In some embodiments, the composition is applied in a volume of about 50 μL to about 300 μL to the skin in a single actuation. In some embodiments, the composition is applied in a volume of about 100 μL in a single actuation. In some embodiments, the composition is applied in a volume of about 50 μL in a single actuation.

The topical compositions provided herein form a protective, washable and breathable film and thus, does not need to be covered after application to the skin. In some embodiments, the topical composition is applied to exposed parts of the body, e.g., the arms. In some embodiments, the topical composition is applied to an area of the skin that does need to be covered with clothing or occlusive items such as bandages.

In some embodiments, the present disclosure provides for topical compositions that require a reduced frequency of administrations for treatment of conditions described herein. For example, in some embodiments, the composition is administered not more than once in a 24 hour period to alleviate symptoms of the conditions described herein.

All references cited herein, including patents, patent applications, papers, textbooks and the like, and the references cited therein, to the extent that they are not already, are hereby incorporated herein by reference in their entirety.

### EXAMPLES

### Example 1—Developing Topical Compositions of Oxybutynin

Compositions 4, 5, 6 and 7 in Table 1 comprising oxybutynin were prepared according to a manufacturing process described herein. Compositions 1-3 and 8-30 are prepared according to a manufacturing process described herein. To prepare the compositions, ethanol and isopropyl alcohol and/or dimethyl sulfoxide were mixed in a suitable vessel to produce a solvent mixture. The film forming excipients, ethacrylic acid and methyl methacrylate copolymer 1:1, methacrylic acid and methyl methacrylate copolymer 1:2, poly(butylmethacrylate-co-(2-dimethylaminoethyl)methacrylate-co-methyl methacrylate 1:2:1, hypromellose, hydroxypropyl cellulose, or ethyl cellulose, and polyethylene glycol 400 were added to the solvent mixture, and the mixture was stirred until a clear solution formed. The penetration enhancers, 1-dodecylazacycloheptan-2-one, isopropyl myristate, octisalate, oleic acid, diethylene glycol monoethyl ether (Transcutol® P), and/or a fragrance were then added to the mixture and the mixture was further stirred. The active ingredient oxybutynin was then added to the mixture and the mixture was again stirred until a clear solution was achieved. Lastly, the solution was added to a suitable container and a spray pump was affixed to the container. Exemplary oxybutynin spray compositions are shown below.

TABLE 1

| | Composition | | | | | | |
|---|---|---|---|---|---|---|---|
| | 1 | 2 | 3 | 4 | 5 | 6 | 7 |
| Ingredient | % w/v | | | | | | |
| Oxybutynin | 1.0 | 2.0 | 3.0 | 4.0 | 6.0 | 8.0 | 10.0 |
| Ethanol USP | 56.0 | 56.5 | 53.9 | 57.0 | 56.0 | 51.0 | 52.1 |
| Isopropyl | 30.0 | 29.5 | 27.4 | 25.5 | 25.5 | 25.0 | 25.5 |
| Alcohol USP | | | | | | | |
| Octisalate USP | 4.0 | 6.0 | 7.0 | 5.0 | 6.0 | 7.0 | 6.0 |
| Eudragit ® S100 | 7.0 | 5.0 | 7.0 | 7.0 | 5.0 | 7.0 | 5.0 |
| USP/NF | | | | | | | |
| Polyethylene | 1.5 | 1.0 | 1.2 | 1.5 | 1.0 | 1.5 | 0.9 |
| glycol 400 | | | | | | | |
| Fragrance | 0.5 | 0.0 | 0.5 | 0.0 | 0.5 | 0.5 | 0.5 |
| TOTAL | 100.0 | 100.0 | 100.0 | 100.0 | 100.0 | 100.0 | 100.0 |

TABLE 2

| | Composition | | | | | | |
|---|---|---|---|---|---|---|---|
| | 8 | 9 | 10 | 11 | 12 | 13 | 14 |
| Ingredient | % w/v | | | | | | |
| Oxybutynin | 2.0 | 4.0 | 6.0 | 6.66 | 8.0 | 20.0 | 10.00 |
| Ethanol USP | 55.0 | 54.4 | 50.9 | 53.84 | 53.7 | 41.5 | 52.1 |
| Isopropyl | 20.5 | 19.1 | 17.4 | 15.5 | 15.8 | 15.5 | 15.5 |
| Alcohol USP | | | | | | | |
| Dimethyl- | 10 | 10 | 10 | 10 | 10 | 10 | 10 |
| sulphoxide | | | | | | | |
| USP | | | | | | | |
| Transcutol ® P | 4.0 | 6.0 | 7.0 | 5.0 | 6.0 | 4.0 | 6.0 |
| USP | | | | | | | |
| Eudragit ® | 7.0 | 5.0 | 7.0 | 7.0 | 5.0 | 7.0 | 5.0 |
| E100 USP/NF | | | | | | | |
| Polyethylene | 1.5 | 1.0 | 1.2 | 1.5 | 1.0 | 1.5 | 0.9 |
| glycol 400 | | | | | | | |
| Fragrance | 0.0 | 0.5 | 0.5 | 0.5 | 0.5 | 0.5 | 0.6 |
| TOTAL | 100.0 | 100.0 | 100.0 | 100.0 | 100.0 | 100.0 | 100.0 |

TABLE 3

| | Composition | | | | | | |
|---|---|---|---|---|---|---|---|
| | 15 | 16 | 17 | 18 | 19 | 20 | 21 |
| Ingredient | % w/v | | | | | | |
| Oxybutynin | 1.0 | 2.0 | 4.0 | 6.00 | 8.00 | 10.00 | 20.00 |
| USP | | | | | | | |
| Ethanol USP | 46.0 | 48.5 | 44.9 | 44.5 | 45.5 | 40.0 | 20.0 |
| Isopropyl | 30.0 | 29.5 | 27.4 | 25.5 | 25.5 | 26.0 | 25.5 |
| Alcohol USP | | | | | | | |
| Oleic Acid | 15.0 | 15.0 | 15.0 | 15.0 | 15.0 | 15.0 | 25.0 |
| USP | | | | | | | |
| Eudragit ® | 7.0 | 4.0 | 7.0 | 7.0 | 5.0 | 7.0 | 7.0 |
| E100 | | | | | | | |
| USP/NF | | | | | | | |
| Polyethylene | 1.5 | 1.0 | 1.2 | 1.5 | 1.0 | 1.5 | 2.0 |
| glycol 400 | | | | | | | |
| Fragrance | 0.5 | 0.0 | 0.5 | 0.5 | 0.0 | 0.5 | 0.5 |
| TOTAL | 100.0 | 100.0 | 100.0 | 100.0 | 100.0 | 100.0 | 100.0 |

TABLE 4

| | Composition | | | | | | |
|---|---|---|---|---|---|---|---|
| | 22 | 23 | 24 | 25 | 26 | 27 | 28 |
| Ingredient | % w/v | | | | | | |
| Oxybutynin | 0.6 | 0.6 | 1.2 | 1.7 | 2.0 | 3.0 | 5.0 |
| USP | | | | | | | |
| Ethanol USP | 58.1 | 58.2 | 54.2 | 59.4 | 60.7 | 55.9 | 56.9 |
| Isopropyl | 29.1 | 29.1 | 29.1 | 25.4 | 25.2 | 25.7 | 25.5 |
| Alcohol USP | | | | | | | |
| Octisalate | 4.0 | 6.0 | 7.0 | 5.0 | 6.0 | 7.0 | 6.0 |
| USP | | | | | | | |
| Eudragit ® | 7.0 | 5.0 | 7.0 | 7.0 | 5.0 | 7.0 | 5.5 |
| S100 USP/NF | | | | | | | |
| Polyethylene | 1.2 | 1.1 | 1.5 | 1.5 | 1.1 | 1.4 | 1.1 |
| Glycol 300 | | | | | | | |
| TOTAL | 100.0 | 100.0 | 100.0 | 100.0 | 100.0 | 100.0 | 100.0 |

TABLE 5

| | Composition | | | | | |
|---|---|---|---|---|---|---|
| | 29 | 30 | 31 | 32 | 33 | 34 |
| Ingredient | % w/v | | | | | |
| Oxybutynin USP | 0.1 | 0.1 | 0.1 | 0.2 | 0.2 | 0.2 |
| Ethanol USP | 58.6 | 58.8 | 55.4 | 61.1 | 62.7 | 59.2 |
| Isopropyl Alcohol | 29.1 | 29.1 | 29.1 | 25.2 | 25.2 | 25.2 |
| USP | | | | | | |
| Octisalate USP | 4.0 | 6.0 | 7.0 | 5.0 | 6.0 | 7.0 |
| Eudragit ® E100 | 7.0 | 5.0 | 7.0 | 7.0 | 5.0 | 7.0 |
| USP/NF | | | | | | |
| Polyethylene | 1.2 | 1.0 | 1.4 | 1.5 | 0.9 | 1.4 |
| Glycol 300 | | | | | | |
| TOTAL | 100.0 | 100.0 | 100.0 | 100.0 | 100.0 | 100.0 |

TABLE 6

| | Composition | | | | | |
|---|---|---|---|---|---|---|
| | 35 | 36 | 37 | 38 | 39 | 40 |
| Ingredient | % w/v | | | | | |
| Oxybutynin USP | 1.7 | 1.7 | 1.7 | 2.0 | 2.0 | 2.0 |
| Ethanol USP | 57.1 | 57.2 | 53.7 | 59.3 | 60.9 | 57.4 |
| Isopropyl Alcohol | 29.0 | 29.1 | 29.2 | 25.2 | 25.2 | 25.2 |
| USP | | | | | | |
| Octisalate USP | 4.0 | 6.0 | 7.0 | 5.0 | 6.0 | 7.0 |
| Eudragit ® S100 | 7.0 | 5.0 | 7.0 | 7.0 | 5.0 | 7.0 |
| USP/NF | | | | | | |
| Polyethylene | 1.2 | 1.0 | 1.4 | 1.5 | 0.9 | 1.4 |
| Glycol 300 | | | | | | |
| TOTAL | 100.0 | 100.0 | 100.0 | 100.0 | 100.0 | 100.0 |

### Example 2—Analysis of Skin Permeation of Oxybutynin Film Forming Compositions

The degree of permeation of oxybutynin film forming compositions into skin were tested. Franz diffusion cells with a donor area of 4.9 cm² and 20 mL receptor chamber volume were assembled with excised abdominal porcine skin. The receptor chamber was filled with phosphate buffered saline (PBS) at pH 7.4. For each experiment, 60 μl of either a 4% w/v or 6% w/v oxybutynin film forming composition (Composition 4 or 5 of Table 1) was added to the donor chamber of the cell.

The samples were maintained at 37° C. and the receptor chamber solution was magnetically stirred to maintain homogeneity. Samples of receptor medium were collected at 1, 2, 4, 6, and 24 hour timepoints post addition of the oxybutynin compositions to the cells and then analyzed by HPLC to determine the amount of oxybutynin permeated through the skin layers.

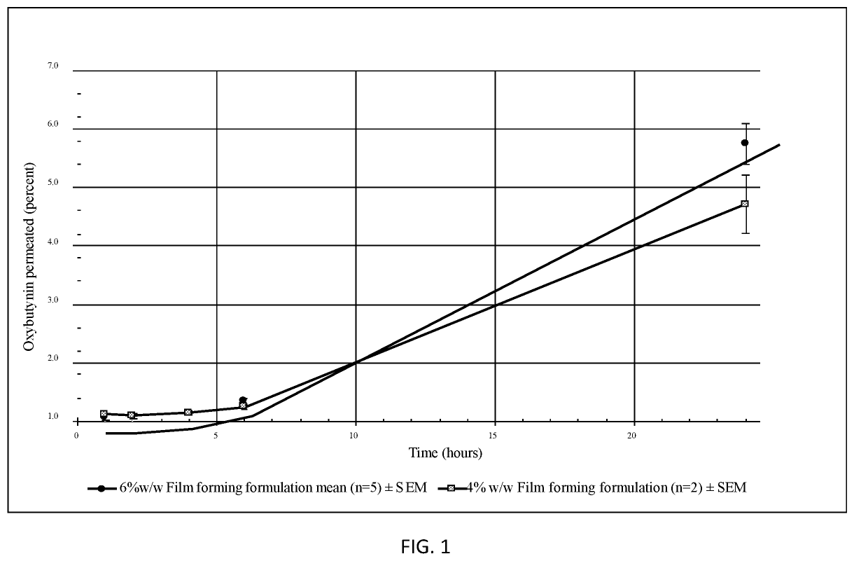

As shown in FIG. 1, oxybutynin permeated through the skin in less than 2 minutes for both the 4% w/v and 6% w/v oxybutynin film forming compositions. The permeation profiles of both compositions show that oxybutynin permeated through the skin over 24 hours.

### Example 3—Analysis of Film Breathability of the Oxybutynin Film Forming Compositions

The film formed after application of the oxybutynin composition is designed to remain on the skin for at least 24 hours. It is desirable that moisture vapor is able to pass through the film. A breathable film ensures that the underlying skin remains in good condition, preventing the development of erythema and other adverse local reactions to the skin. The breathability of the film was tested using the procedure below.

The 8% w/v oxybutynin film forming composition (Composition 6 of Table 1) sample film was applied to the surface of porous surgical tape (Transpore™, 3M, St. Paul, MN) membrane at 12.5 μl per cm² and allowed to dry. A water vapor impermeable container with an opening at the top was partially filled with water. The composition was applied to the porous tape, and the tape with the composition applied was placed over the opening of the container. The container was stored at 37° C. in the presence of calcium chloride desiccant, which maintained a low humidity within the chamber. Water loss from the container over time was determined gravimetrically by measuring the container weight over a period of at least 48 hours. A water vapor transmission rate per area was then determined. Two reference control evaluations were also used in this experiment, a non-occluded (non-film forming) control applied to only the porous tape, and an occluded control applied to porous tape which was covered with non-porous packaging tape. Both controls were applied to the surface of porous surgical tape (Transpore™, 3M, St. Paul, MN) membrane at 12.5 μl per cm² and allowed to dry.

The relative transmission was derived from equation

$$\frac{( Sample Mean Rate - Occluded Control Mean Rate )}{\begin{matrix}( Non - occluded Control Mean Rate - \\ Occluded Control Mean Rate )\end{matrix}} .$$

The sample oxybutynin film had a mean water vapor transmission rate of 1743 g/m² per 24 hours. The rate of mean water vapor transmission as a fraction relative to the non-occluded control was 0.80.

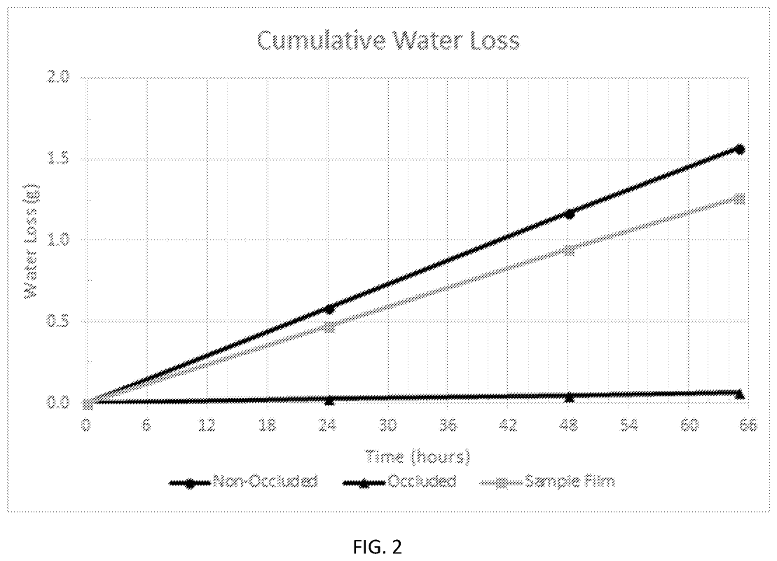

The high-water vapor transmission rate relative to the non-occluded control indicates that the oxybutynin film is not obstructing water vapor transmission and thus maintains the skin in healthy condition. The rate of water loss for each composition over a period of 48 hours is shown in FIG. 2.

### Example 4—Analysis of Drug Transfer of the Oxybutynin Film Forming Compositions

A skin contact transfer study was conducted to compare the active ingredient transfer from the skin after application of the 4% film forming oxybutynin composition (Composition 4 from Table 1) and 30 mg of an oxybutynin chloride 10% gel composition from Gelnique®. Each composition was applied to a glass coupon and allowed to dry for 15 minutes. A polyester swab was firmly pressed against the application area on the coupon. The swab was moved back and forth in overlapping passes over the entire area. The swab passes were repeated alternating the coupon orientation and swab side covering the area for a total of 8 times. The residue on the swab was extracted in 10 mL ethanol and the recovered solutions were analyzed by HPLC. The recovered amounts of oxybutynin were calculated by comparison to a standard of known concentrations.

The transferred residues for the oxybutynin film forming composition were 0.5±0.3% (mean±SD, n=6) of the applied dose. The transferred residues for the oxybutynin gel composition (Gelnique®) were 84.0±9.0% (mean±SD, n=6) of the applied dose. These results indicate that the oxybutynin spray formulation forms an effective barrier to secondary transfer. The film forming composition acts in a similar manner to a clothing barrier to prevent drug transfer.

Additional tests were performed to test the effectiveness of the barrier film. A second application of the film forming composition was sprayed on top of the previously applied spray comprising the formed barrier film. There was no permeation of oxybutynin from the second application of the spray through the barrier film and to the underlying skin. These results indicate that the barrier film is effective in preventing movement of the active ingredient or access to the skin underneath.

### Example 5—Analysis of Film Washability of the Oxybutynin Film Forming Compositions

Washability studies were conducted to determine the washability of the film formed after application of the film forming composition to the skin. The 6% oxybutynin film forming composition (Composition 5 of Table 1) was applied to excised porcine abdominal skin and allowed to dry for 15 minutes. The skin was washed by wiping with a wet soft sponge that was periodically dipped in warm water for 1 minute. The skin was then swabbed with an alcohol wipe. An unwashed control was prepared by wiping the skin surface with only an alcohol wipe to remove the film without washing. The residual oxybutynin on the wipes was quantified.

Recovery of oxybutynin from the wipe used after washing the skin surface with water showed that 0.5% of the applied dose was recovered from the skin surface. Recovery of oxybutynin from the wipe used on the unwashed control sample showed that 84% of the applied dose was recovered from the wipe. These results indicate that the film formed post application of the composition is effectively removed from the skin via washing with water.

### Example 6—Analysis of Crystallization in Film Forming Composition

The 6% oxybutynin film forming composition (Composition 5 of Table 1) was evaluated for any crystallization of oxybutynin. The 6% oxybutynin composition was prepared according to the manufacturing process as described herein. The compositions were then analyzed visually under the microscope for evidence of oxybutynin crystals.

| Composition | Observation |
|---|---|
| 6% oxybutynin film forming composition | No crystals formed |

1. A composition comprising
a. about 1% w/v to about 20% w/v of oxybutynin;
b. a penetration enhancer;
c. at least 65% w/v of an aliphatic solvent; and
d. a film forming excipient,
wherein the film forming excipient has a solubility in water, at a pH between 1 and 10 and wherein the composition is a sprayable liquid solution that forms a washable film when sprayed on skin

2. A composition comprising
a. about 8% w/v of oxybutynin;
b. a penetration enhancer;
c. about 35% to about 45% w/v of an aliphatic solvent; and
d. a film forming excipient,
wherein the film forming excipient has a solubility in water, at a pH between 1 and 10 and wherein the composition is a sprayable liquid solution that forms a washable film when sprayed on skin.

3. The composition of claim 1, wherein the penetration enhancer comprises 1-dodecylazacycloheptan-2-one, isopropyl myristate, octisalate, oleic acid, diethylene glycol monoethyl ether (Transcutol® P), or combinations thereof.

4. The composition of claim 1, wherein the solvent comprises acetone, ethanol and/or isopropyl alcohol.

5. The composition of claim 1, wherein the film forming excipient comprises a polyacrylate polymer or a cellulose polymer.

6. The composition of claim 1, wherein the film forming excipient is methacrylic acid and methyl methacrylate copolymer 1:1, methacrylic acid and methyl methacrylate copolymer 1:2, poly(butylmethacrylate-co-(2-dimethylaminoethyl)methacrylate-co-methyl methacrylate 1:2:1, hypromellose, hydroxypropyl cellulose, and ethyl cellulose.

7. The composition of claim 1, wherein the composition comprises about 4% w/v to about 7% w/v film forming excipient.

8. The composition of claim 1, wherein the penetration enhancer is octisalate, and the film forming excipient is methacrylic acid and methyl methacrylate copolymer 1:1 or the film forming excipient is poly(butylmethacrylate-co-(2-dimethylaminoethyl)methacrylate-co-methyl methacrylate 1:2:1).

9. The composition of claim 1, wherein the composition further comprises a washability enhancer.

10. The composition of claim 9, wherein the washability enhancer is polyethylene glycol 400.

11-20. (canceled)

21. A method of treating hot flashes, hyperhidrosis, osmiodrosis, incontinence, bladder spasms, pain following bladder surgery or combinations thereof in a human, the method comprising applying the composition of claim 1 as a spray to the skin of the human.

22. (canceled)

23. A method of treating moderate to severe vasomotor symptoms caused by menopause in a human female, the method comprising applying the composition of claim 1 as a spray to the skin of the human female.

24-35. (canceled)

36. The method of claim 21, wherein the composition comprises about 0.5% w/v to about 15% w/v oxybutynin.

37. (canceled)

38. The method of claim 21, wherein the volume of the composition applied to the skin in a single actuation is about 500 μL or less.

39-43. (canceled)

44. A composition comprising oxybutynin, octisalate, methacrylic acid and methyl methacrylate copolymer 1:2 or poly(butylmethacrylate-co-(2-dimethylaminoethyl)methacrylate-co-methyl methacrylate 1:2:1) and polyethylene glycol 400.

45-51. (canceled)

52. A spray container comprising
a. the composition of claim 44, and
b. a metering valve.

53. The spray container of claim 52, wherein the spray container further comprises a dose indicator.

54. A method of treating hot flashes, hyperhidrosis, osmiodrosis, incontinence, bladder spasms, pain following bladder surgery or combinations thereof in a human, the method comprising applying the composition of claim 44 as a spray to the skin of the human.

55. (canceled)

56. A method of treating moderate to severe vasomotor symptoms caused by menopause in a human female, the method comprising applying the composition of claim 44 as a spray to the skin of the human female.

57-68. (canceled)

69. The method of claim 54, wherein the composition comprises about 0.5% w/v to about 15% w/v oxybutynin.

70-76. (canceled)
